(19) United States
(12) Patent Application Publication
(10) Pub. No.: US 2015/0224045 A1
(43) Pub. Date: Aug. 13, 2015

(54) RADIATION CURABLE NAIL PREPARATIONS, POST-PROCESSING NAIL FINISHER AND METHODS OF APPLICATION

(71) Applicant: Cali Chem, Inc., Garden Grove, CA (US)

(72) Inventor: Amy Doan, Garden Grove, CA (US)

(73) Assignee: Cali Chem, Inc., Garden Grove, CA (US)

(21) Appl. No.: 14/619,762

(22) Filed: Feb. 11, 2015

Related U.S. Application Data
(60) Provisional application No. 61/938,583, filed on Feb. 11, 2014.
(60) Provisional application No. 62/023,122, filed on Jul. 10, 2014.

(51) Int. Cl. A61K 8/81; A61K 8/35; A45D 29/11; A61K 8/34; A61K 8/37; A61Q 3/02; A61K 8/55

(52) U.S. Cl. CPC A61K 8/8147; A61Q 3/02; A61K 8/35; A61K 8/55; A61K 8/347; A61K 8/37; A61K 8/34; A45D 29/11; A61K 2800/81; A61K 2800/884; A61K 2800/43; A61K 2800/592

(57) ABSTRACT

Preparations for radiation curable nail coatings, finishing preparations for radiation curable nail coatings, and methods of applying such radiation curable nail coatings and finishing preparations are provided. The radiation curable nail coatings and finishing preparations being designed to improve at least one of the tackiness and gloss of the coated nail without the use of a protective topcoat, and methods of applying the preparation also being provided.

### CROSS-REFERENCE TO RELATED APPLICATIONS

The application claims priority to U.S. Provisional Patent Application No. 61/938,583, filed, Feb. 11, 2014, and U.S. Provisional Patent Application No. 62/023,122, filed Jul. 10, 2014, the disclosures of which are incorporated herein by reference.

### FIELD OF THE INVENTION

The present invention relates to radiation curable nail preparations post-application cleansers, such as finishers, and methods of finishing the same. The invention also relates to methods of applying such preparations, and post-application cleansers for such preparations.

### BACKGROUND OF THE INVENTION

Light or radiation curable preparations for use on nails have been known for many years. In these formulations ultraviolet (UV) lamps are used to seal the coated nail (a process called ‘curing’). These radiation curable preparations are favored over traditional acrylic nail preparations, because they can last for a significantly longer period of time, in some cases weeks longer. Partially as a result any different formulations have been described, based on a variety of radiation curable materials, such as, for example, aliphatic hydrocarbon urethane diacrylate and methacrylates (such as are described in U.S. Pat. No. 4,704,303), acrylate urethane oligomers that are crosslinked on curing with UV light (such as are described in U.S. Pat. No. 4,682,612), and more recently bisphenol A diglycidyl methacrylates (‘BISGMA’) (such as are described in U.S. Pat. No. 6,803,394).

Despite the number of different types of formulations, to date the application of such UV curable nail preparations typically require a time-consuming multi-step process. For example, most preparations require not only the cleaning of the underlying nail (whether natural or artificial) by filing, buffing, washing, etc., but additionally requires the application of a base coat to prepare the nail for the application of the coating or artificial nail, and ensure good binding of the coating or artificial nail to the nail substrate. Then, after the coating or artificial nail has been applied and UV cured, a topcoat is applied to provide a hardened protective outer layer to protect the colored nail from damage and wear. Such a process can take thirty minutes or more to apply, set and dry making it inconvenient for the wearer and nail professional.

Some attempts have been made to reduce the complexity of these application processes, and a few radiation curable nail preparations have been introduced that are referred to as ‘one-step’ processes. However, these systems do not provide the sheen and gloss people expect from such preparations, and typically multiple steps are required including the application of a topcoat to reproduce the gloss obtainable from conventional three-step preparations, and a finisher to remove the tackiness of the coated nail. Although these work arounds may ultimately produce satisfactory results, they again add to the complexity of these application processes, and often introduce additional unforeseen problems. Accordingly, a need exists for processes of improving the final appearance of such radiation curable nail preparations.

### SUMMARY OF THE INVENTION

Radiation curable nail preparations, finishing preparations for radiation curable nail and/or artificial nail tips, and methods of finishing such radiation curable nail and/or artificial nail tips are described.

In some embodiments the invention is directed to a radiation curable nail preparation curable under exposure to a radiative source including:
between about 1 and 35% by weight of a pigment;
between about 0.05 and 8% by weight of a solvent selected from the group;
between about 0.05 and 6% of a photoinitiator;
between about 45 and 80% of a polymeric photochemical system comprised of between about 50 and 98% by weight of a polymerizable material and a photoinitiator;
wherein the polymeric photochemical system is curable under a radiative source to form a nail coating; and
wherein the nail coating is compatible with a nail finishing preparation substantially comprising at least one oil material such that the glossiness of the nail coating is maintained in the absence of a top protective coating.

In other embodiments the nail preparation further includes between about 0.5 and 8% by weight of a silicone oil.

In still other embodiments the nail preparation includes between about 50 and 75% by weight of the polymeric photochemical system.

In yet other embodiments the nail finishing preparation includes between about 30 and 80% by weight of an oil material and a solvent containing no isopropyl alcohol, wherein the finishing preparation improves at least the gloss of the nail coating absent a top protective coating.

In still yet other embodiments the photochemical system includes up to 5% by weight of a photoinitiator, and a polymeric material including between about 45 and 80% by weight of a urethane oligomer and between 5 and 30% by weight of a (meth)acrylated monomer, and further including up to 1% by weight of an inhibitor and 0.5 to 10% by weight of a solvent.

In still yet other embodiments the polymeric photochemical system further includes additives selected from the group consisting of secondary photoinitiators, photoaccelerators, fillers, preservatives, inhibitors, plasticizers, colorants, dyes, oxygen scavengers, optical brighteners, dispersion aids, waxes, non-reactive polymers, adhesion promoting monomers or polymers, nanofillers, organsols, fibers, and solvents.

In still yet other embodiments the polymerizable material of the polymeric photochemical system includes at least an urethane oligomer and a (meth)acrylate monomer.

In still yet other embodiments the polymeric photochemical system includes:
at least one polymerizable material selected from the group consisting of acrylates and methacrylates, urethane methacrylates, aliphatic polyols, aromatic polyols, polyester polyols, and polyether polyols, aliphatic polyurethanes, aromatic polyurethanes, polyester polyurethanes, polyether diisocyanates, polyurethane acrylate oligomers di-Hema trimethylhexyl dicarbamate, polyol methacrylates, 2-hydroxyethyl methacrylate, hydroxypropyl methacrylate, and mixtures thereof;
at least 5 to 20% by weight of at least one photoinitiator selected from the group consisting of ketones, benzyl ketones, sulfanyl ketones, monomeric hydroxyl ketones, polymeric hydroxyl ketones, phosphinates, acyl phosphine oxides, metallocenes, benzophenone and derivatives, hydroxycyclohexylphenylketone, trimethylbenzoylphosphine oxide, and mixtures thereof;
at least one inhibitor selected from the group consisting of hydroquinone and a p-hydroxyanisole;
a colorant; and
at least 1 to 10% by weight of at least one solvent selected from carbonates, propylene carbonate, acetone, acetates, ethyl acetate, butyl acetate, toluene, alcohols, and isopropyl alcohol.

In still yet other embodiments the polymeric photochemical system includes:
a polymerizable material including 50-75% by weight of a polyurethane acrylate oligomer, 10-25% by weight of a 2-hydroxyethyl methacrylate, and 10-25% by weight of a hydroxypropyl methacrylate;
a photoinitiator including at least one of 0.1-1% by weight of a hydroxycyclohexyl phenyl ketone and 0.1-1% by weight of a trimethylbenzoyl diphenylphosphine oxide;
an inhibitor comprising at least one of up to 0.005% by weight of a hydroquinone, and 0-0.02% by weight of a p-hydroxyanisole;
up to 0.1 by weight of a colorant; and
a solvent including 2-10% by weight of butyl acetate, ethyl acetate, and isopropyl alcohol.

In still yet other embodiments the polymeric photochemical system includes:
a polymerizable material includes 45-55% by weight of a polyurethane acrylate oligomer, 15-20% by weight of a 2-hydroxyethyl methacrylate, and 15-20% by weight of a hydroxypropyl methacrylate;
a photoinitiator includes at least one of 0-1% by weight of a hydroxycyclohexyl phenyl ketone and 0-1% by weight of a trimethylbenzoyl diphenylphosphine oxide;
an inhibitor includes at least one of 0-0.005% by weight of a hydroquinone, 0-0.02% by weight of a p-hydroxyanisole;
up to 1% by weight of a colorant; and
a binder includes 0-15% by weight of polyethylene glycol 400 dimethacrylate.

In still yet other embodiments the photoinitiator includes at least one photoinitiator selected from the group of phosphines, ketones, azides, and phosphate salts, and mixtures thereof.

In still yet other embodiments the solvent includes at least one solvent selected from the group of carbonates, acetone, acetates, toluene, and alcohols.

In some other embodiments the invention is directed to a finishing preparation for a cured radiation curable nail materials including:
between about 30 and 80% by weight of an oil material;
an isopropyl alcohol free solvent; and
wherein the finishing preparation enhances the glossiness of a nail coating formed of a cured radiation curable nail material in the absence of a top coat by at least 2% when compared with the nail coating formed of the cured radiation curable nail material in the absence of a top coat finished without the finishing preparation.

In still other embodiments the oil material is 30% by weight of caprylic/capric triglyceride, and the solvent is 69% by weight of ethanol.

In yet other embodiments the oil material is selected from a group consisting of animal oil, vegetable oil, coconut oil, glyceride, triglyceride, caprylic/capric triglyceride and silicone.

In still yet other embodiments the finishing preparation includes additives selected from the group consisting of preservatives, colorants, and fragrances.

In still yet other embodiments the finishing preparation comprises 2 to 10 by weight of an additive.

In still yet other embodiments the glossiness of the nail coating is enhanced by at least 5%.

In still yet other embodiments the glossiness of the nail coating is enhanced by at least 20%.

In still other embodiments the invention is directed to methods of applying a radiation curable nail finishing preparation including:
applying a coating of a radiation curable nail material directly atop a nail wherein the radiation curable nail material includes:
   between about 1 and 35% by weight of a pigment;
   between about 0.05 and 8% by weight of a solvent selected from the group;
   between about 0.05 and 6% of a photoinitiator;
   between about 45 and 80% of a polymeric photochemical system including between about 50 and 98% by weight of a polymerizable material and a photoinitiator;
exposing the radiation curable material to a radiative source for a time suitable to cure the material and form a nail coating; and
applying a finishing preparation including between about 30 and 80% by weight of an oil material atop the radiation curable nail material, wherein the finishing preparation enhances the glossiness of the nail coating by at least 2% when compared with a nail coating formed of the cured radiation curable nail material in the absence of a top coat finished without the finishing preparation.

In some such embodiments the radiation curable nail preparation is (meth)acrylate based.

In still other such embodiments the method further includes buffing the finished nail coating with a low grit buffer.

In yet other such embodiments the method further includes applying an adhesive base coat material to the surface of the nail prior to applying a coating of the radiation curable nail material.

In still yet other such embodiments the nail is one of either a natural or artificial nail.

In yet other such embodiments the glossiness of the nail coating is enhanced by at least 20%.

In yet other embodiments the invention is directed to a method of applying a radiation curable nail preparation including:
providing a nail coating formed of a radiation curable nail material applied directly atop a nail and having no top coat applied thereto; and
applying a finishing preparation comprising between about 30 and 80% by weight of an oil material atop the radiation curable nail material, wherein the finishing preparation enhances the glossiness of the nail coating by at least 2% when compared with a nail coating formed of the cured radiation curable nail material in the absence of a top coat finished without the finishing preparation.

In some such embodiments the method includes buffing the finished nail coating with a low grit buffer.

In other such embodiments the method includes applying an adhesive base coat material to the surface of the nail prior to applying a coating of the radiation curable nail material.

In still other such embodiments the nail is one of either a natural or artificial nail.

In yet other such embodiments the glossiness of the nail coating is enhanced by at least 20%.

Other objects, advantages and novel features, and further scope of applicability of the present invention will be set forth in part in the detailed description to follow, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE DRAWINGS

The description will be more fully understood with reference to the following figures, which are presented as exemplary embodiments of the invention and should not be construed as a complete recitation of the scope of the invention, wherein:

FIG. 1 illustrates a flow chart of a method for finishing a radiation curable nail material atop a natural nail in accordance with embodiments of the present invention;

FIG. 2 illustrates a flow chart of a method for finishing a radiation curable nail material atop an artificial nail/nail tip in accordance with embodiments of the present invention; and

FIG. 3 illustrates a data graph providing the results of a glossiness study conducted on embodiments of finishing materials in accordance with the present invention.

### DETAILED DESCRIPTION OF THE INVENTION

Turning now to the data and description, preparations for radiation curable nail preparations, preparations for finishing a nail having applied thereto a radiation curable nail preparation, methods of applying such radiation curable nail preparations and methods of applying such finishers are provided. In many embodiments the radiation curable nail preparations to be finished comprise a ‘single-step’ formulation that allows for the application of the preparation without the requirement that separate base and top coats also be applied. In some embodiments the method of applying the single-step radiation curable nail preparation includes the use of a finishing preparation, such preparation improving at least one of the tackiness and/or gloss of the coated nail. In other embodiments the finishing preparations and methods improve at least the gloss of the coated nail when compared to coated nails formed using similar non top coated radiation curable preparations by at least 2% and up to at least 30%.

### DEFINITIONS

It will be understood that term “preparation” or “material”, in singular and/or plural, may substitute for composition, such as a composition of matter.

The term “(meth)acrylate” as used herein, means methacrylate, acrylate, or mixtures thereof.

The term “nail” as used herein, will be understood to mean either finger or toe nails.

The terms “polymeric” or “polymeric materials” as used throughout the specification and claims is intended to include resins, monomers, oligomers, and polymers.

Percentages listed in the disclosure are in weight % unless indicated otherwise.

All ranges cited in this disclosure (even where they sum to 100%) are understood to include substitutions by appropriate amounts of standard nail preparation additives, including, but not inclusive of, solvents, preservatives, coupling agents, plasticizers, secondary photoinitiators, photoaccelerators, colorants, dyes, inhibitors, oxygen scavengers, optical brighteners, dispersion aids, waxes, fillers, nanofillers, organsols, fibers, and adhesion promoting monomers or polymers, or other additives known in the art.

Unless otherwise indicated all percentage measurements are “by weight”.

Throughout the disclosure, the term ‘single-step’ refers to a nail coating material that can be applied in a single application without the use of a base or top coat.

### Background Discussion of Radiation Curable Gel Preparations

Conventional radiation curable gel nail preparations systems, typically incorporate three different materials:
a base coat, made of a clear or milky-colored polish and perhaps fortified to contain proteins, vitamin E, calcium and other such ingredients, that is designed to help the coating adhere to the nail and/or used to enhance the health of the nails (examples of base coat materials may include solvents, hydrogenated rosins and (meth)acrylate oligomers);
a clear or colored gel varnish; and
a top coat, which is applied after the gel has been applied to protects the nail color from scratches, chipping and peeling, and to strengthen the nails and maintain a shiny and glossy appearance in the nail coating, which otherwise might chip or become dull and tacky.

While these multi-step gel systems give adequate results, and have been extensively used, they are time-consuming and complicated to apply. In particular, the time required to apply and then let each layer within the process set sufficiently can take well more than an hour. This makes the use of such gel systems less attractive.

Some manufacturers have attempted to address this fundamental problem by introducing systems that combine elements of the conventional three-step gel preparations. For example, many manufacturers have either combined the base coat and color varnish, or color varnish and topcoat into single products. Again, while these have shown adequate results, they still require substantial time for application. Some manufacturers have attempted to introduce single-step systems in which base coat smoothing, color and topcoat hardener functionalities are all incorporated into a single preparation. However, thus far the results have not been satisfactory. In particular, many of these preparations remain tacky and easy to peel off (because of the lack of a base coat), and lack the high gloss and sheen (because of the lack of a top coat) of conventional multi-step preparations, even after application of conventional finishers.

### Discussion of Inventive Radiation Curable Nail Preparations

In various embodiments the radiation curable nail preparations are based on a combination of a pigment, a solvent, and a polymeric photochemical system that includes a photo initiator and a mixture of one or more polymerizable compounds, these materials being selected to be compatible with a finisher for removing the tackiness of the underlying nail preparation and to ensuring the gloss and sheen of the final prepared nail.

In some embodiments the radiation curable nail preparation comprises between about 1 and 35% (by weight) of a pigment, between about 0.05 and 8% of a solvent, between about 0.05 and 6% of a photoinitiator, between about 0.5 and 8% of a silicone oil, and the remainder being of a polymeric photochemical material. In other embodiments, the radiation curable nail preparation comprises between about 2 and 31% of a pigment, between about 0.1 and 4% of a solvent, between about 0.1 and 3% of a photoinitiator, between about 1 and 4% of a silicone oil, and the remainder being of a polymeric photochemical material. In yet other embodiments, the radiation curable nail preparation comprises between about 3 and 30% of a pigment, between about 0.1 and 4% of a solvent, between about 0.1 and 3% of a photoinitiator, between about 1 and 4% of a silicone oil, and the remainder being of a polymeric photochemical material. In still yet other embodiments, the polymeric photochemical material comprises between about 45 and 80% of the preparation, and in still yet other embodiments between about 50 and 75% of the preparation.

The Polymeric Photochemical System of the Radiation Curable Nail Material

Turning first to the polymeric photochemical system or polymeric photochemical material, in many embodiments the photochemical material may include one or more polymerizable materials, and one or more photoinitiators and photoaccelerators, along with other conventional additives.

In many embodiments, the polymeric photochemical material comprises between approximately 50 and approximately 98 percent by weight, and preferably between approximately 70 and approximately 95 percent by weight of polymerizable material; and between approximately 0.05 and approximately 10 percent by weight, preferably between approximately 0.2 and approximately 2 percent by weight of one or more photoinitiators; and additionally, the polymeric photochemical material may include photo accelerators such as aliphatic or aromatic amines, as well as fillers, preservatives, inhibitors, plasticizers, non-reactive polymers, adhesion promoters and solvents.

In some embodiments the polymeric photochemical material comprises a urethane oligomer, one or more (meth)acrylated monomers, one or more photoinitiators/accelerators, one or more inhibitors, one or more solvents and a colorant. In one embodiment, the composition comprises: between approximately 45 and 80 percent by weight, preferably between approximately 50 and 75 percent by weight, and more preferably between about 45 and 55 percent by weight of a urethane oligomer; between approximately 5 and 30 percent by weight, preferably between approximately 10 and 25 percent by weight, and more preferably between about 15 and 20 weight percent of at least one and in many embodiments two (meth)acrylated monomers; between approximately 0 and 5 percent by weight, preferably between approximately 0.1 and 1 percent by weight, of at least one photoinitiator; between approximately 0 and 1 percent by weight, preferably between approximately 1 and 0.1 percent by weight, of at least one inhibitor, and between approximately 0.5 and 10 percent by weight, preferably between approximately 1 and 5 percent by weight, of at least one solvent.

In many embodiments, the polymerizable material may incorporate (meth)acrylate-based polymerizable monomers, as well as other polymerizable monomers, oligomers or polymers of monomers. Some embodiments of such materials include acrylates and (meth)acrylates, including, urethane(meth)acrylates, including aliphatic, aromatic, polyester, and polyether polyols and aliphatic, aromatic, polyester based polyurethanes, and polyether diisocyanates capped with (meth)acrylate endgroups. In some embodiments the polymerizable materials includes a mixture of at least a polyurethane acrylate oligomer (such as, for example, di-Hema trimethylhexyl dicarbamate), and one or more polyol methacrylates, such as, for example, 2-hydroxyethyl methacrylate (HEMA) and hydroxypropyl methacrylate.

The polymeric photochemical material may also contain one or more photoinitiators, including, for example, ketones (including benzyl ketones, sulfanyl ketones, monomeric hydroxyl ketones, polymeric hydroxyl ketones, etc.), phosphinates (including acyl phosphine oxides), metallocenes, and benzophenone and derivatives. In some embodiments, the polymerizable material may include hydroxycyclohexylphenylketone and trimethylbenzoylphosphine oxide (TPO), and mixtures thereof.

The polymeric photochemical material may also contain one or more photoaccelerators, such as for example, aliphatic amines and/or aromatic amines, such as, for example an aminobenzoate, amino (meth)acrylate.

The polymeric photochemical material may also contain a coupling agent, such as, for example, an organo-metallic such as an organo-titanate.

In addition to the above, the polymeric photochemical material may optionally include plasticizers, additional photoinitiators, colorants, solvents, dyes, preservatives, inhibitors, fillers, fibers, and/or adhesion promoting polymers. Examples of such additive materials include, but are not inclusive of: preservatives, plasticizers (e.g., phthalates, adipates, and/or sulfonamides), additional photoinitiators (e.g., quinone derivatives, dimethylketal derivatives, and or benzophenone), colorants (e.g., but not limited to, barium, calcium and aluminum lakes, iron oxides, chromates, molybdates, cadmiums, metallic or mixed metallic oxides, talcs, carmine, titanium dioxide, chromium hydroxides, ferric ferrocyanide, ultramarines, titanium dioxide coated mica platelets, and/or bismuth oxychlorides), inhibitors (e.g., hydroquinones, and/or anisoles), fillers (e.g., mineral and/or polymeric fillers), fibers, adhesion promoters (e.g., phthalates), binders (e.g., polyethylene glycol 400 dimethacrylate), and solvents (e.g., alcohols and/or acetates).

The composition ranges for some embodiments of a polymeric photochemical material is set forth in Table 1, below.

TABLE 1
Polymeric Photochemical Composition Ranges

| | | % (by |
|---|---|---|
| Class of Materials | Compositions | weight) |
| Polymerizable | Polyurethane acrylate oligomer | 50-75 |
| materials | 2-hydroxyethyl methacrylate | 10-25 |
| | Hydroxypropyl methacrylate | 10-25 |
| Photoinitiator/ | Hydroxycyclohexyl phenyl ketone | 0.1-1 |
| accelerators | Trimethylbenzoyl diphenyl- | 0.1-1 |
| | phosphine oxide | |
| Inhibitors | Hydroquinone | 0-0.005 |
| | p-hydroxyanisole | 0-0.02 |
| Colorants | (Any Suitable) | 0-0.1 (4) |
| Solvents | Butyl acetate, ethyl acetate, | 2-10 |
| | isopropyl alcohol | |

The composition ranges for other embodiments of a polymeric photochemical material is set forth in Table 2, below.

TABLE 2
Polymeric Photochemical Composition Ranges

| | | % (by |
|---|---|---|
| Class of Materials | Compositions | weight) |
| Polymerizable | Polyurethane acrylate oligomer | 45-55 |
| materials | 2-hydroxyethyl methacrylate | 15-20 |
| | Hydroxypropyl methacrylate | 15-20 |
| Photoinitiator/ | Hydroxycyclohexyl phenyl ketone | 0-1 (4) |
| accelerators | Trimethylbenzoyl diphenyl- | 0-1 (4) |
| | phosphine oxide | |
| Inhibitors | Hydroquinone | 0-0.005 |
| | p-hydroxyanisole | 0-0.02 |
| Colorants | (Any Suitable) | 0-1 (4) |
| Binder | Polyethylene glycol 400 | 0-15 |
| | dimethacrylate | |

Regardless of the specific formulation used in making the polymeric photochemical material, the material should be curable under a radiation source, such as a UV lamp.

The Photoinitiators of the Radiation Curable Nail Material

Turning now to the photoinitiators, preferably the radiation curable nail preparation includes at least one photoinitiator in an amount of from about 5 to 20%, preferably from about 10 to 13%. In some embodiments the photoinitiator comprises at least one photoinitiator selected from the group of phosphines, (including phosphinates and phosphine oxides), ketones (including, sulfanyl ketones and polymeric morpholinoketones), azides (including sulfonyl azides), and phosphate salts (including iodonium hexafluorophosphate), and mixtures thereof. One exemplary photoinitiator mixture is PL-3100™, manufactured by Palermo Lundahl Industries.

The Solvents of the Radiation Curable Nail Material

Turning now to the solvents, preferably the radiation curable nail preparation includes at least one solvent in an amount of from about 1 to 10%, preferably from about 2 to 5%. In some embodiments the solvent comprises at least one solvent selected from the group of carbonates (including propylene carbonate), acetone, acetates (including ethyl and butyl acetate), toluene, and alcohols (including isopropyl alcohol).

The Pigments of the Radiation Curable Nail Material

Turning now to the pigments, preferably the radiation curable nail preparation includes at least one, but potentially many, pigments including barium, calcium and aluminum lakes, iron oxides, chromates, molybdates, cadmiums, metallic or mixed metallic oxides, talcs, carmine, titanium dioxide, chromium hydroxides, ferric ferrocyanide, ultramarines, titanium dioxide coated mica platelets, and/or bismuth oxychlorides. Specific pigment formulations include D&C Black Nos. 2 & 3, FD&C Blue Nos. 1 & 4, D&C Brown No. 1, FD&C Green Nos. 3, 5, 6 & 8, D&C Orange Nos. 4, 5, 10 and 11, FD&C Red Nos. 4, 6, 7, 17, 21, 22, 27, 28, 30, 31, 33, 34, 36 and 40, D&C Violet No. 2, FD&C Yellow Nos. 5, 6, 7, 8, 10 and 11, as well as any others approved for use in cosmetics applications. Although dingle pigments are described above, it should be understood that the pigments can include mixtures of many pigments, such as for example, COLORSPERSE RED®, manufactured by Gayson Silicone Dispersions, Inc.;

The above noted pigments may be provided pure or dispersed in a carrier liquid comprised of one or more materials such as, for example, solvents. Examples of suitable solvents include acetates (such as, for example, butyl acetate & ethyl acetate), alcohols (such as, for example, isopropanol, ethanol, etc.), other organic compounds (such as, for example, xylene, toluene, acetone, and ketones). The pigments may be dispersed in the solvents or other materials (such as oligomers or monomers) by any means, for example by mixing or shearing of the pigment directly with the other materials. The carrier liquid may also comprise polymers (such as, for example, cellulose-based polymers and other non-reactive polymers), fillers, and dispersants. Regardless of the ultimate pigment formulation, the pigment is homogenously dispersed into the carrier liquid and mixture of pigment and carrier incorporated into the final radiation curable nail material by blending.

Suitable Silicone Oils for the Radiation Curable Nail Material

Finally, in addition to the above, the radiation curable nail material may include silicone oils, such as, for example, a siloxane, including polydimethylsiloxane or dimethicone.

Suitable Additives for the Radiation Curable Nail Material

Finally, in addition to the above, the radiation curable nail material may optionally include plasticizers, additional photoinitiators, colorants, solvents, dyes, preservatives, inhibitors, fillers, fibers, and/or adhesion promoting polymers. Examples of such additive materials include, but are not inclusive of: preservatives, plasticizers (e.g., phthalates, adipates, and/or sulfonamides), inhibitors (e.g., hydroquinones, and/or anisoles), fillers (e.g., mineral and/or polymeric fillers), fibers, and adhesion promoters (e.g., phthalates).

Again, although specific materials and formulations of radiation curable nail materials are provided above, in all cases the material is curable under exposure to a radiative source such as visible or UV light. Preferably, the composition may cure, in preferred embodiments, in less than approximately ten minutes with exposure to a radiative source, such as an actinic (UV or short wavelength source).

In addition, in many embodiments the radiation curable material is designed for use with a specific finishing material, the finishing material for use in removing the tackiness of the cured radiation curable nail material, and to preserve the gloss and shine of the prepared nail.

### Method of Applying the Radiation Curable Nail Material

Although the above discussion has focused on radiation curable nail materials and systems, it should be understood that other embodiments are directed to methods of applying the radiation curable nail materials. Unlike conventional radiation curable nail materials that require application of a base and/or top coat, the radiation curable nail materials set forth in embodiments may be applied without resort to these additional steps, as will be discussed in greater detail below.

### Application to Natural Nails

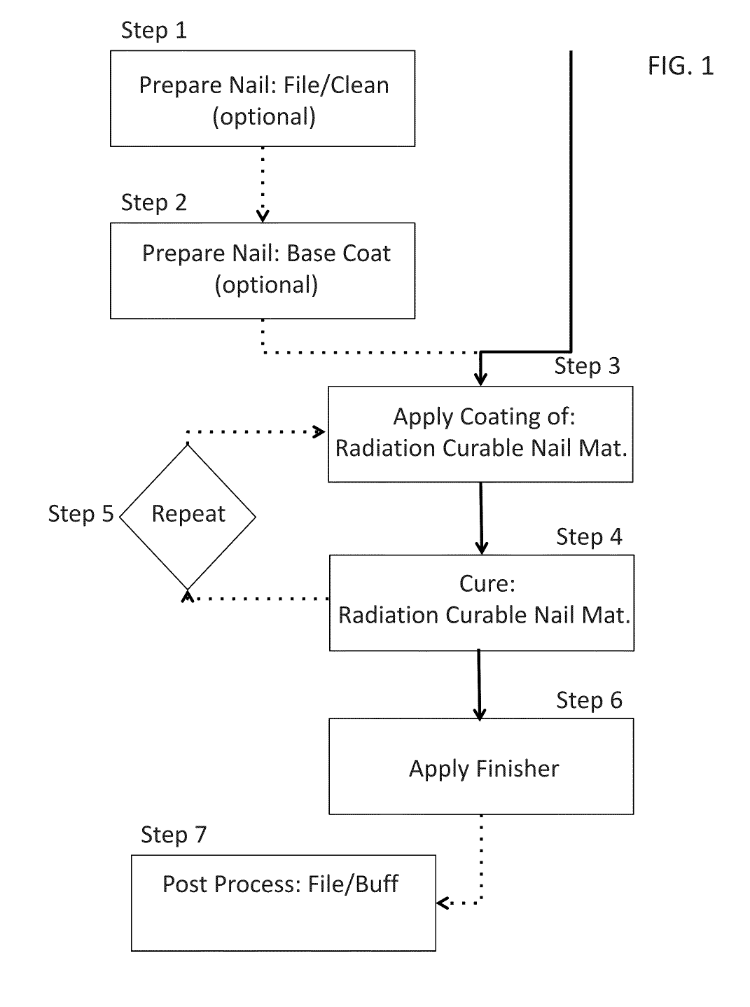

In use on a natural nail, the radiation curable nail materials may be applied directly to the nail. The natural nails are typically prepared by filing, and then one or more coats of the radiation curable nail material is/are applied. Although not necessary, it is to be understood that optionally a thin coat of a solvent-based adhesive base coat may also be applied to the surface of the natural nail. The radiation curable nail material may then be applied to this adhesive base coat. The steps of such a procedure is shown in the flow chart provided in FIG. 1, and described below.

As shown in FIG. 1, some embodiments are directed to a method of creating a coated nail using a radiation curable nail material on a natural nail including the following steps:
Step 1: File natural nail to remove oil and shine to minimize any subsequent lifting (optional);
Step 2: Select and apply at least one coat of an adhesive base coat bond agent (optional);
Step 3: Select and apply one coating of a radiation curable nail material;
Step 4: Expose the radiation curable nail material to a radiative source for a time suitable to cure the material;
Step 5; Repeat steps 3 & 4 as required;
Step 6: Remove any residue from the coated nails with a finishing preparation; and
Step 7: Post-application processing, such as buffing, filing or the application of a high sheen top coating (optional).

As provided in the flowchart of FIG. 1, in many embodiments, the radiation curable nail material may be applied to a natural nail. In such embodiment, the nail may be optionally filed on the top surface to remove oils and create a surface for bonding. Again, although not necessary for the operation of the radiation curable nail materials of the disclosure, atop this untreated nail an adhesive base coat material may also optionally be applied to part or the entire surface of the nail. Once any optional preparatory steps are completed a first radiation curable nail material is applied to the nail and cured via exposure to a radiative source. The application of multiple such coatings or partial coatings of the same or different radiation curable nail materials may be made to create the final nail effect desired. For example, if a French manicure look is desired a first white coat or white tip coat of radiation curable nail material may be applied and cured, followed by a clear or colored coating of such material, which would then be cured. Again, although not necessary to the operation of the radiation curable nail material, the coated nail may then be optionally filed and buffed and a clear top coating material applied. An optional second coat or multiple clear coats may be applied. Once all of the layers of radiation curable material are applied and cured, a finishing material is used to remove any tackiness and to give the nail a high gloss shine. Although one specific method is described, other colors of coatings are anticipated and may be used in any number of combinations or alone.

Application to Artificial Nails/Nail Tips

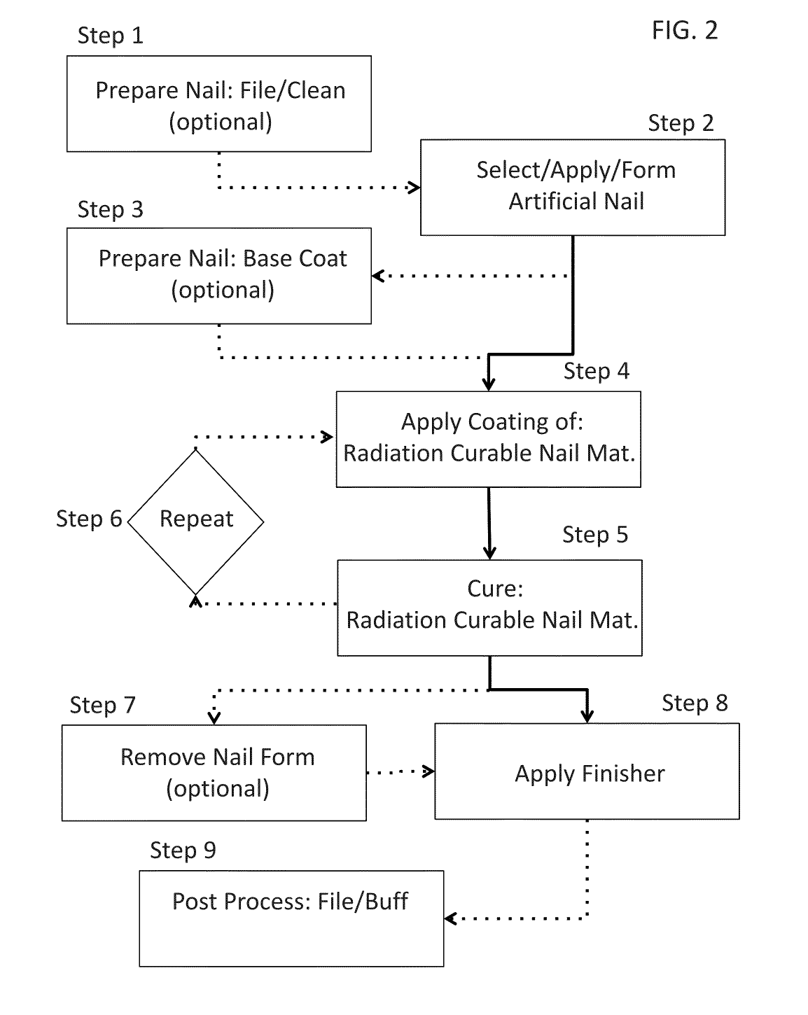

Although the above discussion has focused on embodiments of radiation curable nail preparations and processes for their application, it will be understood that in some embodiments the preparations may be used in association with pre-formed artificial nail tips. Such nail tips may be of any suitable manufacture, and are conventionally formed from injection molded plastics, e.g., ABS, nylon and acetate. In embodiments where the preparations are used in conjunction with artificial nail tips, the artificial nail would first be applied to the underlying natural nail, such as through the use of a suitable adhesive. Once applied, the artificial nail might then be prepared, such as by filing to blend the artificial nail tip into the fingernail and/or to prepare the surface of the artificial nail for the application of the radiation curable nail preparation. The steps of such a procedure is shown in the flow chart provided in FIG. 2, and described below.

As shown in FIG. 2, some embodiments are directed to a method of creating a coated nail using a radiation curable nail material in association with an artificial nail/nail tip, including the following steps:
Step 1: File natural nail to remove oil and shine to minimize any subsequent lifting (optional);
Step 2: Select a nail tip and/or nail form, apply the nail tip/form using an adhesive (e.g., glue or radiation curable nail material), shape nail tip/firm, blend nail tip/form to natural nail;
Step 3: Select and apply at least one coat of an adhesive base coat bond agent (optional);
Step 4: Select and apply one coating of a radiation curable nail material;
Step 5: Expose the radiation curable nail material to a radiative source for a time suitable to cure the material;
Step 6; Repeat steps 4 & 5 as required;
Step 7: If a nail form was applied then remove the nail form.
Step 8: Remove any residue from the coated nails with a finishing preparation; and
Step 9: Post-application processing, such as buffing, filing or the application of a high sheen top coating (optional).

While the above discussion assumes that the artificial nail/nail tip would be attached to the underlying nail using a glue or other adhesive, it should be understood that the artificial nail/nail tip might also be applied to the underlying nail using the radiation curable nail preparation.

### Discussion of Inventive Finisher Preparations

Many embodiments are directed to a “finishing” preparation for application to the cured radiation curable nail preparation, and in particular to finishing materials designed to enhance the polished characteristics (gloss/sheen/tackiness) of a coated nail or nail tip. While finishing preparations of the type generally proposed are known and have been used with other nail coating materials, it has been discovered that finishing single-step preparations, of the type set forth herein, poses special challenges. In particular, it has been discovered that most finishing preparations do not work to remove the tackiness and provide the type of gloss and sheen obtained with multi-step nail gel coatings when used with conventional single-step gel coatings. This has resulted in the need for the application of top-coatings or the use of other post-application processes that damages the utility of these conventional single step coatings.

It has now been discovered that special formulations of oils, when used in combination with particular embodiments of the (meth)acrylate based radiation curable nail materials set forth in the embodiments herein, work to remove tackiness and produce a high sheen, high gloss nail finish without the need for any additional post-application processing, such as the use of high sheen top coat materials, or mechanical manipulation of the nail, such as, for example, filing and buffing.

In many embodiments, the finishing material comprises at least one solvent and an oil, and in some embodiments a natural oil (e.g., animal and/or vegetable oil) or a silicone oil (e.g., a siloxane). In some such embodiments finishing preparations are provided for radiation curable nail materials where a glossy finish is desired that incorporate glyceride and triglyceride materials. In such embodiments, for example, the oil may comprise a glyceride or triglyceride containing material or mixture. In one embodiment the finishing material comprises: between approximately 00 and approximately 80 percent by weight, preferably between about 40 and 60 percent by weight, and more preferably about 50% of a solvent; and between approximately 30 and approximately 80 percent by weight, preferably about 40 and 60 by weight, and more preferably about 50 percent by weight, of an oil or oil containing material. Turning to the composition of the finishing material, in many embodiments the solvent comprises any suitable solubilizing material such as, for example, acetone, ethyl alcohol, ethyl acetate, and/or methyl ethyl ketone.

The use of some solvents in the finishing preparations, such as isopropyl alcohol, can have a negative effect on the finish of the nail. Accordingly, in many embodiments the finishing preparation does not include a solvent including an isopropyl alcohol.

The finishing material also comprises a suitable oil, such as, for example, a glyceride containing oil such as coconut oil. In some embodiments the finishing material includes a solution of glycerides, such as caprylic/capric triglyceride, decanoyl, octanoyl, or mixtures thereof in a solvent, such as, for example, ethanol. Other formulations of finishing preparation may require less buffing. For example, in another particular embodiment the finishing preparation may contain 69% ethanol, 30% caprylic/capric triglyceride and 1% fragrance. In this embodiment, buffing is not essential for enhancing gloss.

In other embodiments directed to finishing preparations for radiation curable nail materials where a matte finish is desired the preparations incorporate silicone oils. In such embodiments, for example, the oil may comprise a substantially pure silicone oil, such as for example, a siloxane. In many embodiments the siloxane is a polydimethylsiloxane, such as, for example, a dimethicone, such as XIAMETER® manufactured by Dow Corning.

In addition, any of the finishing materials described may further include preservatives, colorants and/or fragrances, preferably in an amount from about 2 to 10 percent by weight. One suitable material may include, for example, LEXOL®, manufactured by Inolex Chemical Company or ENDIMATE®, manufactured by Coast Southwest, Inc.

As will be discussed in greater detail, in many embodiments the inventive finishing material demonstrates an improvement in the glossiness of a coated nail formed by a radiation curable nail material absent a top coat when compared with similarly coated nails finished with other conventional finishing materials, such as alcohol based finishers. In some such embodiments the glossiness is improved by at least 2%, in other embodiments by at least 5%, in still other embodiments by at least 20%, and in still other embodiments by at least 34%. Such improvement in the glossiness of the finished radiation curable nails is unexpected, and demonstrates that across different types of radiation curable materials (applied without a top coat) embodiments of the finishing materials taught herein provide a level of glossiness previously unobtainable.

### Method of Finishing the Radiation Curable Nail Material

Although the above discussion has focused on finishing materials for radiation curable nail materials and systems, it should be understood that other embodiments are directed to methods of applying and finishing the radiation curable nail materials. Unlike conventional radiation curable nail materials that require application of a base and/or top coat, the radiation curable nail finisher materials set forth in embodiments may be applied without resort to these additional steps, as will be discussed in greater detail below, and yet still act to maintain the gloss of the finished nail and prevent tackiness.

### Application to Natural Nails

In use on a natural nail, the radiation curable nail finishing materials may be applied directly to a natural nail that has been coated with a radiation curable nail material. The natural nails are typically prepared by filing, and then one or more coats of the radiation curable nail material is/are applied. Although not necessary, it is to be understood that optionally a thin coat of a solvent-based adhesive base coat may also be applied to the surface of the natural nail. The radiation curable nail material may then be applied to this adhesive base coat. Regardless of the preparation and application of the radiation curable nail material, the finishing material according to embodiments is applied directly atop the radiation curable nail material without intermediate application of a top coat. The steps of such a procedure are shown in the flow chart provided in FIG. 1, and described below.

As shown in FIG. 1, some embodiments are directed to a method of finishing a coated nail using a finishing material atop a radiation curable nail material coated on a natural nail including the following steps:
Step 1: File natural nail to remove oil and shine to minimize any subsequent lifting (optional);
Step 2: Select and apply at least one coat of an adhesive base coat bond agent (optional);
Step 3: Select and apply one coating of a radiation curable nail material;
Step 4: Expose the radiation curable nail material to a radiative source for a time suitable to cure the material;
Step 5; Repeat steps 3 & 4 as required;
Step 6: Remove any residue from the coated nails and enhance the gloss with a finishing preparation; and
Step 7: Post-application processing, such as buffing, filing or the application of a high sheen top coating (optional).

As provided in the flowchart of FIG. 1, in many embodiments, the radiation curable nail material may be applied to a natural nail. In such embodiment, the nail may be optionally filed on the top surface to remove oils and create a surface for bonding. Again, although not necessary for the operation of the radiation curable nail materials of the disclosure, atop this untreated nail an adhesive base coat material may also optionally be applied to part or the entire surface of the nail. Once any optional preparatory steps are completed a first radiation curable nail material is applied to the nail and cured via exposure to a radiative source. The application of multiple such coatings or partial coatings of the same or different radiation curable nail materials may be made to create the final nail effect desired. For example, if a French manicure look is desired a first white coat or white tip coat of radiation curable nail material may be applied and cured, followed by a clear or colored coating of such material, which would then be cured. An optional second coat or multiple clear coats may be applied. Again, the order and content of the above steps are not necessary to the operation of the finisher material. In all embodiments, regardless of order of method, once the radiation curable nail material is applied and cured, a finishing material, according to embodiments, is applied atop the radiation curable nail material. The finishing material capable of removing any tackiness and to give the nail a high gloss shine. The finished nail may then be optionally buffed. It should be understood that in such buffing any suitable very low grit buffer designed for polishing and shining the nail may be used, such as, for example, a chamois cloth, electric buffer or foam buffer block, such as are sold under the name Sand Sponge Block™ sold by Soft Touch®, or under the name I Love Buffers Innovative Buffers™. Although one specific method is described, other colors of coatings are anticipated and may be used in any number of combinations or alone.

### Application to Artificial Nails/Nail Tips

Although the above discussion has focused on embodiments of radiation curable nail finishing preparations and processes for their application, it will be understood that in some embodiments the finishing preparations may be used in association with pre-formed artificial nail tips. Such nail tips may be of any suitable manufacture, and are conventionally formed from injection molded plastics, e.g., ABS, nylon and acetate. In embodiments where the finishing preparations are used in conjunction with artificial nail tips, the artificial nail would first be applied to the underlying natural nail, such as through the use of a suitable adhesive. Once applied, the artificial nail might then be prepared, such as by filing to blend the artificial nail tip into the fingernail and/or to prepare the surface of the artificial nail for the application of the radiation curable nail preparation. The steps of such a procedure are shown in the flow chart provided in FIG. 2, and described below.

As shown in FIG. 2, some embodiments are directed to a method of finishing a coated nail using a radiation curable nail finishing material in association with an artificial nail/nail tip, including the following steps:
Step 1: File natural nail to remove oil and shine to minimize any subsequent lifting (optional);
Step 2: Select a nail tip and/or nail form, apply the nail tip/form using an adhesive (e.g., glue or radiation curable nail material), shape nail tip/firm, blend nail tip/form to natural nail;
Step 3: Select and apply at least one coat of an adhesive base coat bond agent (optional);
Step 4: Select and apply one coating of a radiation curable nail material;
Step 5: Expose the radiation curable nail material to a radiative source for a time suitable to cure the material;
Step 6: Repeat steps 4 & 5 as required;
Step 7: If a nail form was applied then remove the nail form.
Step 8: Remove any residue from the coated nails and enhance the gloss with a finishing preparation; and
Step 9: Post-application processing, such as buffing, filing or the application of a high sheen top coating (optional).

While the above discussion assumes that the artificial nail/nail tip would be attached to the underlying nail using a glue or other adhesive, it should be understood that the artificial nail/nail tip might also be applied to the underlying nail using the radiation curable nail preparation.

### Exemplary Embodiments

Enhancing the Finish of Nails Prepared with the Radiation Curable Nail Material

As discussed above, many embodiments are directed to preparations and methods for enhancing the polished characteristics (gloss/sheen/tackiness) of a coated nail or nail tip. In one embodiment, the method comprises the steps of: applying a finishing preparation to a nail coated with a cured radiation curable nail material as described above. The finishing preparation may be applied by any suitable means, such as, for example, with a cotton swab or pad, or sprayed directly onto the nail. The nail may then optionally be rubbed or buffed, for example, with a clean cloth.

In some embodiments the finishing material comprises at least one solvent and an oil, and in some embodiments a natural oil (e.g., animal and/or vegetable oil). In such an embodiment, for example, the natural oil comprises coconut oil. In one embodiment the finishing material comprises: between approximately 00 and approximately 80 percent by weight, and preferably about 50% of a solvent; and between approximately 30 and approximately 80 percent by weight, preferably about 50 percent by weight, of an oil or oil containing material. Turning to the composition of the finishing material, in many embodiments the solvent comprises any suitable solubilizing material such as, for example, acetone, ethyl alcohol, ethyl acetate, isopropyl alcohol, and/or methyl ethyl ketone. However, it should be noted that in certain embodiments containing isopropyl alcohol and colorant, these finishing preparations may remove pigment and dull finish. The finishing material also comprises a suitable oil, such as, for example, coconut oil. In one particular embodiment the finishing material includes a solution of caprylic/capric triglyceride in a solvent, such as, for example, ethanol. In addition, the finishing material may further include preservatives, colorants and/or fragrances. One suitable material may include, for example, LEXOL®, manufactured by Inolex Chemical Company.

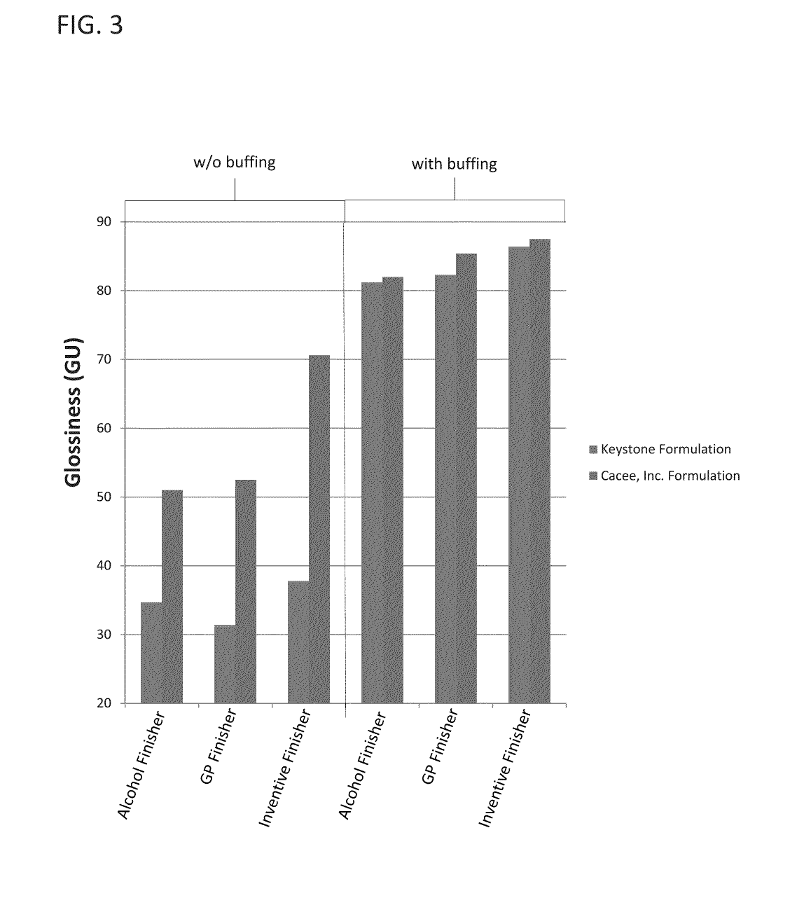

Using such methods and preparations, nails have been prepared and tested using a ETB-0686 Glossmeter Instrument (having a measure range of 0-200 gloss units (GU), a stability of <±0.4 GU/30 Min, a value error of <±1.2 GU and a power of 1.5V*2, set at an angle of 60°) in accordance with ISO2813 to demonstrate the efficacy of the preparations and methods described herein. In one test a finishing material according to embodiments comprising about 80% by weight of a silicone oil was applied to a nail being coated atop two different radiation curable nail preparations manufactured by Keystone® and Cacee, Inc.®, and the shine and gloss tested as described above with the glossmeter, both before and after using a buffing procedure. For comparison, samples were also finished on the same radiation curable nail preparations using a standard alcohol based cleanser sold under the name Gel Polish™ manufactured by Sally Hansen®, both before and after buffing with a shiny buffer, and also using a regular alcohol cleanser (99% isopropyl alcohol), before and after buffing with a shiny buffer. The results of these gloss studies are summarized in Table 1, below, and summarized in the data plots provided in FIG. 3. (It should be noted that these tests were conducted over time to ensure that the gloss level provided by the various tested finishing materials was persistent.)

TABLE 3
Glossiness Test Results

| | Inventive | Inventive | | GP | | Alcohol |
|---|---|---|---|---|---|---|
| | Oil | Oil Finisher | GP | Finisher | Alcohol | Finisher |
| Nail | Finisher | (w/buffing) | Finisher | (w/buffing) | Finisher | (w/buffing) |
| Preparation | (GU) | (GU) | (GU) | (GU) | (GU) | (GU) |
| Keystone ® | 37.8 | 86.4 | 31.4 | 82.3 | 34.7 | 81.2 |
| Cacee, Inc. ® | 70.6 | 87.5 | 52.5 | 85.4 | 51 | 82 |

As demonstrated, in all of the results, either alone or with buffing, the inventive finishing material provides a persistent level of gloss to the radiation curable nail preparations that is far superior to either the GP Finisher, or the conventional alcohol finisher, either with or without buffing. Indeed, embodiments of the inventive finishing material demonstrate a 20 to 34% improvement in the glossiness of the final nail when compared to the GP finisher, and a 2 to 9% improvement when compared to the conventional alcohol finisher when buffing is not applied. With buffing, embodiments of the inventive finishing materials demonstrate a 5 to 6% improvement in the glossiness of the final nail when compare to the GP finisher, and a 6 to 7% improvement when compared to the conventional alcohol finisher. Such improvement in the glossiness of the finished radiation curable nails is completely unexpected, and demonstrates that across different types of radiation curable materials (applied without a top coat) embodiments of the finishing materials taught herein provide a level of glossiness and gloss protection unattainable with conventional finishing materials and methods.

### Doctrine of Equivalents

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above, and of the corresponding application(s), are hereby incorporated by reference.

What is claimed is:

1. A radiation curable nail preparation curable under exposure to a radiative source comprising:
between about 1 and 35% by weight of a pigment;
between about 0.05 and 8% by weight of a solvent selected from the group;
between about 0.05 and 6% of a photoinitiator;
between about 45 and 80% of a polymeric photochemical system comprised of between about 50 and 98% by weight of a polymerizable material and a photoinitiator;
wherein the polymeric photochemical system is curable under a radiative source to form a nail coating; and
wherein the nail coating is compatible with a nail finishing preparation substantially comprising at least one oil material such that the glossiness of the nail coating is maintained in the absence of a top protective coating.

2. The nail preparation of claim 1, further comprising between about 0.5 and 8% by weight of a silicone oil.

3. The nail preparation of claim 1, wherein the nail preparation is comprised of between about 50 and 75% by weight of the polymeric photochemical system.

4. The nail preparation of claim 1, wherein the nail finishing preparation comprises between about 30 and 80% by weight of an oil material and a solvent containing no isopropyl alcohol, wherein the finishing preparation improves at least the gloss of the nail coating absent a top protective coating.

5. The nail preparation of claim 1, wherein the photochemical system comprises up to 5% by weight of a photoinitiator, and a polymeric material comprised of between about 45 and 80% by weight of a urethane oligomer and between 5 and 30% by weight of a (meth)acrylated monomer, and further comprises up to 1% by weight of an inhibitor and 0.5 to 10% by weight of a solvent.

6. The nail preparation of claim 1, wherein the polymeric photochemical system further comprises additives selected from the group consisting of secondary photoinitiators, photoaccelerators, fillers, preservatives, inhibitors, plasticizers, colorants, dyes, oxygen scavengers, optical brighteners, dispersion aids, waxes, non-reactive polymers, adhesion promoting monomers or polymers, nanofillers, organsols, fibers, and solvents.

7. A nail preparation of claim 1, wherein the polymerizable material of the polymeric photochemical system is comprised of at least an urethane oligomer and a (meth)acrylate monomer.

8. A nail preparation of claim 1, wherein the polymeric photochemical system is comprised of:
at least one polymerizable material selected from the group consisting of acrylates and methacrylates, urethane methacrylates, aliphatic polyols, aromatic polyols, polyester polyols, and polyether polyols, aliphatic polyurethanes, aromatic polyurethanes, polyester polyurethanes, polyether diisocyanates, polyurethane acrylate oligomers di-Hema trimethylhexyl dicarbamate, polyol methacrylates, 2-hydroxyethyl methacrylate, hydroxypropyl methacrylate, and mixtures thereof;
at least 5 to 20% by weight of at least one photoinitiator selected from the group consisting of ketones, benzyl ketones, sulfanyl ketones, monomeric hydroxyl ketones, polymeric hydroxyl ketones, phosphinates, acyl phosphine oxides, metallocenes, benzophenone and derivatives, hydroxycyclohexylphenylketone, trimethylbenzoylphosphine oxide, and mixtures thereof;
at least one inhibitor selected from the group consisting of hydroquinone and a p-hydroxyanisole;
a colorant; and
at least 1 to 10% by weight of at least one solvent comprising selected from the group consisting of carbonates, propylene carbonate, acetone, acetates, ethyl acetate, butyl acetate, toluene, alcohols, and isopropyl alcohol.

9. A nail preparation of claim 1, wherein the polymeric photochemical system is comprised of:
a polymerizable material comprising 50-75% by weight of a polyurethane acrylate oligomer, 10-25% by weight of a 2-hydroxyethyl methacrylate, and 10-25% by weight of a hydroxypropyl methacrylate;
a photoinitiator comprising at least one of 0.1-1% by weight of a hydroxycyclohexyl phenyl ketone and 0.1-1% by weight of a trimethylbenzoyl diphenylphosphine oxide;
an inhibitor comprising at least one of up to 0.005% by weight of a hydroquinone, and 0-0.02% by weight of a p-hydroxyanisole;
up to 0.1 by weight of a colorant; and
a solvent comprising 2-10% by weight selected from the group consisting of butyl acetate, ethyl acetate, and isopropyl alcohol.

10. A nail preparation of claim 1, wherein the polymeric photochemical system is comprised of:
a polymerizable material comprising 45-55% by weight of a polyurethane acrylate oligomer, 15-20% by weight of a 2-hydroxyethyl methacrylate, and 15-20% by weight of a hydroxypropyl methacrylate;
a photoinitiator comprising at least one of 0-1% by weight of a hydroxycyclohexyl phenyl ketone and 0-1% by weight of a trimethylbenzoyl diphenylphosphine oxide;
an inhibitor comprising at least one of 0-0.005% by weight of a hydroquinone, 0-0.02% by weight of a p-hydroxyanisole;
up to 1% by weight of a colorant; and
a binder comprised of 0-15% by weight of polyethylene glycol 400 dimethacrylate.

11. A nail preparation of claim 1, wherein the photoinitiator comprises at least one photoinitiator selected from the group of phosphines, ketones, azides, and phosphate salts, and mixtures thereof.

12. A nail preparation of claim 1, wherein the solvent comprises at least one solvent selected from the group of carbonates, acetone, acetates, toluene, and alcohols.

13. A finishing preparation for a cured radiation curable nail materials comprising:
between about 30 and 80% by weight of an oil material;
an isopropyl alcohol free solvent; and
wherein the finishing preparation enhances the glossiness of a nail coating formed of a cured radiation curable nail material in the absence of a top coat by at least 2% when compared with the nail coating formed of the cured radiation curable nail material in the absence of a top coat finished without the finishing preparation.

14. The finishing preparation of claim 13, wherein the oil material is 30% by weight of caprylic/capric triglyceride, and the solvent is 69% by weight of ethanol.

15. The finishing preparation of claim 13, wherein the oil material is selected from a group consisting of animal oil, vegetable oil, coconut oil, glyceride, triglyceride, caprylic/capric triglyceride and silicone.

16. The finishing preparation of claim 13, further comprising additives selected from the group consisting of preservatives, colorants, and fragrances.

17. The finishing preparation of claim 18, further comprising 2 to 10% by weight of an additive.

18. The finishing preparation of claim 18, wherein the glossiness of the nail coating is enhanced by at least 5%.

19. The finishing preparation of claim 18, wherein the glossiness of the nail coating is enhanced by at least 20%.

20. A method of applying a radiation curable nail finishing preparation comprising:
applying a coating of a radiation curable nail material directly atop a nail wherein the radiation curable nail material comprises:
between about 1 and 35% by weight of a pigment;
between about 0.05 and 8% by weight of a solvent selected from the group;
between about 0.05 and 6% of a photoinitiator;
between about 45 and 80% of a polymeric photochemical system comprised of between about 50 and 98% by weight of a polymerizable material and a photoinitiator;
exposing the radiation curable material to a radiative source for a time suitable to cure the material and form a nail coating; and
applying a finishing preparation comprising between about 30 and 80% by weight of an oil material atop the radiation curable nail material, wherein the finishing preparation enhances the glossiness of the nail coating by at least 2% when compared with a nail coating formed of the cured radiation curable nail material in the absence of a top coat finished without the finishing preparation.

21. The method in claim 20, wherein the radiation curable nail preparation is (meth)acrylate based.

22. The method in claim 20, further comprising buffing the finished nail coating with a low grit buffer.

23. The method in claim 20, further comprising applying an adhesive base coat material to the surface of the nail prior to applying a coating of the radiation curable nail material.

24. The method in claim 20, wherein the nail is one of either a natural or artificial nail.

25. The method in claim 20, wherein the glossiness of the nail coating is enhanced by at least 20%.

26. A method of applying a radiation curable nail preparation comprising:
providing a nail coating formed of a radiation curable nail material applied directly atop a nail and having no top coat applied thereto; and
applying a finishing preparation comprising between about 30 and 80% by weight of an oil material atop the radiation curable nail material, wherein the finishing preparation enhances the glossiness of the nail coating by at least 2% when compared with a nail coating formed of the cured radiation curable nail material in the absence of a top coat finished without the finishing preparation.

27. The method in claim 26, further comprising buffing the finished nail coating with a low grit buffer.

28. The method in claim 26, further comprising applying an adhesive base coat material to the surface of the nail prior to applying a coating of the radiation curable nail material.

29. The method in claim 26, wherein the nail is one of either a natural or artificial nail.

30. The method in claim 26, wherein the glossiness of the nail coating is enhanced by at least 20%.
